(19) United States
(12) Patent Application Publication
(10) Pub. No.: US 2008/0262233 A1
(43) Pub. Date: Oct. 23, 2008

(54) SYNTHESIS OF PREFFERED ISOMER FRACTIONS OF N, N-DIETHYLDECAHYDROQUINOLINIUM CATION

(75) Inventor: Allen W. Burton, Richmond, CA (US)

Correspondence Address: CHEVRON CORPORATION, P.O. BOX 6006, SAN RAMON, CA, 94583-0806, US

(73) Assignee: Chevron U.S.A. Inc.

(21) Appl. No.: 11/866,267

(22) Filed: Oct. 2, 2007

Related U.S. Application Data
(60) Provisional application No. 60/829,436, filed on Oct. 13, 2006.

(51) Int. Cl. C07D 215/04

(52) U.S. Cl. 546/181

(57) ABSTRACT

The present invention relates to a process for synthesizing trans-N,N-diethyldecahydroquinolinium cation or a mixture of a cis-N,N-diethyldecahydroquinolinium cation and a trans-N,N-diethyldecahydroquinolinium cation.

This application claims the benefit of provisional Application No. 60/829,436, filed Oct. 13, 2006.

### FIELD OF THE INVENTION

A method for synthesizing trans-NN N-diethyldecahydroquinolinium cation or a mixture of cis-N,N-diethyldecahydroquinolinium cation and trans-N,N-diethyldecahydroquinolinium cation.

### BACKGROUND OF THE INVENTION

A synthesis process for synthesizing N,N-diethyldecahydroquinolinium cation is described in U.S. Pat. No. 6,080,382. That synthesis process does not include steps to treat mixed isomers of N,N-diethyldecahydroquinolinium to obtain preferred isomer fractions. Preferred isomer fractions are desired especially fractions with higher levels of cis-isomers, as these fractions have utility as structure directing agents (SDA) in the synthesis of molecular sieves such as the aluminum-containing molecular sieve SSZ-26.

### SUMMARY OF THE INVENTION

There is provided a process for synthesizing trans-N,N-diethyldecahydroquinolinium cation or a mixture of cis-N,N-diethyldecahydroquinolinium cation and trans-N,N-diethyldecahydroquinolinium cation, comprising:
a. hydrogenating quinoline in the presence of acetic acid, sulfuric acid and a platinum oxide catalyst to form a mixture of cis-decahydroquinoline and trans-decahydroquinoline;
b. recovering the mixture of cis-decahydroquinoline and trans-decahydroquinoline and freezing it;
c. thawing the frozen mixture of cis-decahydroquinoline and trans-decahydroquinoline until it separates into a liquid fraction and a solid fraction;
d. recovering the liquid fraction from step (c);
e. reacting the liquid fraction with ethyl halide to form a solid product which is a mixture of cis-N,N-diethyldecahydroquinolinium cation and trans-N,N-diethyldecahydroquinolinium cation;
f. recovering the solid product of step (e);
g. slurrying the solid product in acetone to selectively recover a precipitate with an increased amount of trans-N,N,-diethyldecahydroquinolinium cation and a remaining oily product recovered from the acetone having an increased amount of cis-N,N-diethyldecahydroquinolinium cation.

There is also provided a method for synthesizing preferred isomer fractions of N N-diethyldecahydroquinolinium cation, comprising:
a. forming a mixture of cis-decahydroquinoline and trans-decahydroquinoline, wherein the mixture has at least 25% cis-decahydroquinoline;
b. freezing and thawing the mixture until it separates into a liquid fraction and a solid fraction, wherein the liquid has greater than 40% cis-decahydroquinoline;
c. reacting the liquid fraction with ethyl halide to form a solid product which is a mixture of cis-N,N-diethyldecahydroquinolinium cation and trans-N,N-diethyldecahydroquinolinium cation;
d. recovering the solid product of step (c);
e. slurrying the solid product in acetone to selectively recover a precipitate with an increased amount of trans-N,N,-diethyldecahydroquinolinium cation and a remaining oily product recovered from the acetone having at least about 60% cis-N,N-diethyldecahydroquinolinium cation.

There is also provided a procedure for synthesizing preferred isomer fractions of N,N-diethyldecahydroquinolinium cation, comprising:
a. freezing and thawing a mixture of cis-decahydroquinoline and trans-decahydroquinoline, wherein the mixture has at least about 25% cis-decahydroquinoline, until the mixture separates into a liquid fraction having greater than about 40% cis-decahydroquinoline and a solid fraction;
b. reacting the liquid fraction with ethyl halide to form a solid product which is a mixture of cis-N,N-diethyldecahydroquinolinium cation and trans-N,N-diethyldecahydroquinolinium cation;
c. recovering the solid product of step (b) by filtering and extracting the filtrate with a halogenated hydrocarbon solvent,
d. slurrying the solid product in acetone to selectively recover a precipitate with an increased amount of trans-N,N, diethyldecahydroquinolinium cation and a remaining oily product recovered from the acetone having at least about 60% cis-N,N-diethyldecahydroquinolinium cation.

### DETAILED DESCRIPTION

The cis-N,N-diethyldecahydroquinolinium cation and trans-N,N-diethyldecahydroquinolinium cation have the following structures:

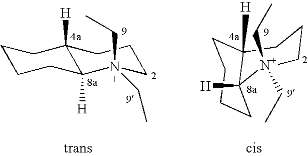

The general scheme for synthesizing trans-N,N-diethyldecahydroquinolinium cation or a mixture of cis-N,N-diethyldecahydroquinolinium cation and trans-N,N-diethyldecahydroquinolinium cation is to use a source of decahydroquinoline that has an appreciable quantity (at least about 40%) of the cis isomer. This amine is then alkylated with an ethyl halide to give a quaternary ammonium salt. Decahydroquinoline is generally prepared by hydrogenation of quinoline with a metal or metal oxide catalyst. The nature of the metal catalyst will affect the cis/trans ratio of the final product.

In one embodiment, the synthesis is conducted as follows:
a. hydrogenating quinoline in the presence of acetic acid, sulfuric acid and a platinum oxide catalyst to form a mixture of cis-decahydroquinoline and trans-decahydroquinoline;
b. recovering the mixture of cis-decahydroquinoline and trans-decahydroquinoline and freezing it;
c. thawing the frozen mixture of cis-decahydroquinoline and trans-decahydroquinoline until it separates into a liquid fraction and a solid fraction;
d. recovering the liquid fraction from step (c);
e. reacting the liquid fraction with ethyl halide to form a solid product which is a mixture of cis-N,N-diethyldecahydroquinolinium cation and trans-N N-diethyldecahydroquinolinium cation;
f. recovering the solid product of step (e);
g. slurrying the solid product in acetone to selectively recover a precipitate with an increased amount of trans-N,N,-diethyldecahydroquinolinium cation and a remaining oily product recovered from the acetone having an increased amount of cis-N,N-diethyldecahydroquinolinium cation.

In a second embodiment the synthesis is conducted as follows:
a. forming a mixture of cis-decahydroquinoline and trans-decahydroquinoline, wherein the mixture has at least 25% cis-decahydroquinoline;
b. freezing and thawing the mixture until it separates into a liquid fraction and a solid fraction, wherein the liquid fraction has greater than 40% cis-decahydroquinoline;
c. reacting the liquid fraction with ethyl halide to form a solid product which is a mixture of cis-N,N-diethyldecahydroquinolinium cation and trans-N,N-diethyldecahydroquinolinium cation;
d. recovering the solid product of step (c);
e. slurrying the solid product in acetone to selectively recover a precipitate with an increased amount of trans-N,N,-diethyldecahydroquinolinium cation and a remaining oily product recovered from the acetone having at least about 60% cis-N,N-diethyldecahydroquinolinium cation.

In a third embodiment the synthesis is conducted as follows:
a. freezing and thawing a mixture of cis-decahydroquinoline and trans-decahydroquinoline, wherein the mixture has at least about 25% cis-decahydroquinoline, until the mixture separates into a liquid fraction having greater than about 40% cis-decahydroquinoline and a solid fraction;
b. reacting the liquid fraction with ethyl halide to form a solid product which is a mixture of cis-N,N-diethyldecahydroquinolinium cation and trans-N,N-diethyldecahydroquinolinium cation;
c. recovering the solid product of step (b) by filtering and extracting the filtrate with a halogenated hydrocarbon solvent;
d. slurrying the solid product in acetone to selectively recover a precipitate with an increased amount of trans-N,N,-diethyldecahydroquinolinium cation and a remaining oily product recovered from the acetone having at least about 60% cis-N,N-diethyldecahydroquinolinium cation.

In one embodiment, the mixture of cis-decahydroquinoline and, trans-decahydroquinoline formed by hydrogenating quinoline in the presence of acetic acid, sulfuric acid and a platinum oxide catalyst. This mixture initially has a content of cis isomers less than 50%. It is desired, however, that the content of cis isomers in the mixture of cis-decahydroquinoline and trans-decahydroquinoline be high; for example, at least about 25% or at least about 40%. Lower ratios of trans/cis isomers of decahydroquinoline are obtained by the freezing and thawing steps. The liquid fraction separated from the freezing and thawing step has an appreciable quantity of the cis isomer (greater than about 40% cis-decahydroquinoline) and in some embodiments is greater than about 50% or about 60% cis-decahydroquinoline.

The mixture of cis-decahydroquinoline and trans-decahydroquinoline is cooled until it is frozen. The frozen product is then thawed and allowed to melt. The thawed product separates into a liquid fraction and a solid fraction. The liquid fraction is recovered and reacted with ethyl halide (e.g., ethyl iodide, ethyl chloride or ethyl bromide) to form a mixture of cis-N,N-diethyldecahydroquinolinium cation and trans-N,N-diethyldecahydroquinolinium cation.

In one embodiment the step of recovering a solid product which is a mixture of cis-N,N-diethyldecahydroquinolinium cation and trans-N,N-diethyldecahydroquinolinium cation includes filtering and extracting the filtrate with a halogenated hydrocarbon solvent. Examples of halogenated hydrocarbon solvents are dichloromethane, chloroform, ethylene dichloride and carbon tetrachloride.

It has been found that the cis-NN-diethyldecahydroquinolinium cation is highly soluble in acetone, whereas the trans-N N-diethyldecahydroquinolinium cation is only sparingly soluble in acetone. Thus, when the solid product of the reaction with ethyl halide is slurried in acetone, the cis isomer dissolves in the acetone while most of the trans isomer does not (some of the trans isomer may dissolve in the acetone along with the cis isomer).

In one embodiment the steps used to selectively recover a precipitate with an increased amount of trans-N,N,-diethyldecahydroquinolinium cation and a remaining oily product recovered from the acetone having an increased amount of cis-N,N-diethyldecahydroquinolinium cation include:
a. removing the acetone from the slurry by filtration;
b. removing the acetone from the filtrate to yield an oil product;
c. adding acetone and ethyl ether to the oil to cause a solid to precipitate; and
d. recrystallizing and recovering the precipitate and the remaining oily product recovered from the acetone having an increased amount of cis-N,N-diethyldecahydroquinolinium cation.

The acetone is removed to selectively recover the precipitate with an increased amount of trans-N,N,-diethyldecahydroquinolinium cation and a remaining oily product having an increased amount of cis-N,N,-diethyldecahydroquinolinium cation from the slurry. In one embodiment the acetone is removed from the slurry by filtration. The acetone is then removed from the filtrate (such as by evaporation) to yield an oily product. Acetone and ethyl ether are added to the oily product which causes a precipitate to form. The precipitate can then be recrystallized and recovered. The remaining oily product is a mixture of cis-N,N-diethyldecahydroquinolinium cation and trans-N,N-diethyldecahydroquinolinium cation. The precipitate is predominantly trans-N,N-diethyldecahydroquinolinium cation, and in some embodiments is at least 80% up to 100% trans-N,N-diethyldecahydroquinolinium cation. The remaining oily product has a reduced trans/cis ratio, generally less than 50/50. In some embodiments the trans/cis ratio is between about 10/90 to about 40/60, or between about 10/90 to about 30/70. The remaining oily product contains at least 50% cis-N,N-diethyldecahydroquinolinium cation, In some embodiments, the remaining oily product has levels of cis-N,N-diethyldecahydroquinolinium cation of at least about 60% or at least about 70%. The level of cis-N,N-diethyldecahydroquinolinium cation is generally less than 95%, and in some embodiments is less than 90%.

The cis-N,N-diethyldecahydroquinolinium cation or mixture of cis-N,N-diethyldecahydroquinolinium cation and trans-N,N-diethyldecahydroquinolinium cation is useful as a structure directing agent for synthesizing the molecular sieve designated SSZ-26.

The following examples demonstrate, but do not limit, the present invention.

### EXAMPLES

### Example 1

### Synthesis of Decahydroquinoline

200 mL glacial acetic acid, 15 mL concentrated sulfuric acid, and 152 g quinoline (1.18 mol) were added to a large stainless steel reactor equipped with a hydrogen flow. Next 15 g of platinum oxide catalyst was added to the mixture. The reaction vessel was then sealed and pressurized and depressurized three times with dry nitrogen. At the end of each depressurization step, the pressure in the reactor vessel was maintained above atmospheric pressure. The reactor was then pressurized with hydrogen gas to 1500 psi and then depressurized to just above atmospheric pressure twice. The vessel was then pressurized to 1500 psi hydrogen. After a few hours, the pressure dropped to 400 psi and the reactor vessel was then pressurized again with hydrogen to 1500 psi. After an additional two hours, the pressure again dropped to about 400 psi. The vessel was again pressurized with hydrogen to 1500 psi, and the react on was allowed to continue overnight. At the end of the reaction, the pressure was constant at about 1400 psi.

At this point, the contents of the reactor were removed and the platinum oxide catalyst was removed by filtration. About 300 mL water was then added to the filtrate solution and then NaOH pellets were added and dissolved in the solution until the pH>12. An organic layer was observed above the aqueous solution upon the increase in pH. The organic product was then extracted from the mixture using ethyl ether. The ether solution was then dried over magnesium sulfate, and the ether was removed by rotoevaporation to yield the desired decahydroquinoline. ¹H and ¹³C liquid NMR indicated the decahydroquinoline product was pure within experimental limits and that it possessed about a 60/40 trans/cis ratio of isomers.

Next, a fraction of the decahydroquinoline product was placed in a round-bottom flask and the flask was then cooled with dry ice until the amine had completely frozen. At this point, the flask was removed from the dry ice and then tilted slightly on its side. Part of the solid then began to thaw. After the mixture had warmed, two separate fractions were formed: a liquid fraction which collected on the bottom of the flask and a mostly solid fraction on the side of the flask. The solid fraction was still slightly wet. NMR of the two fractions indicated the liquid fraction was about 45/55 trans/cis and the solid fraction was about 75/25 trans/cis.

### Example 2

### Synthesis of N,N-Diethyldecahydroquinolinium

In a 500 mL round-bottom flask, 33.2 g (0.24 mol) of the liquid decahydroquinoline fraction was mixed with 228 mL methanol. 34.8 g potassium bicarbonate (0.35 mol) was then added, and a magnetic stirrer was added to the mixture to allow mixing in the subsequent steps. Next, 90.2 g iodoethane (0.58 mol) was added dropwise. After allowing the mixture to stir at room temperature for two hours, the mixture was refluxed overnight. The mixture was then allowed to cool to room temperature, and the potassium salts were removed by filtration. The filtrate was then rotoevaporated to remove the methanol solvent. The resulting solids were then extracted with chloroform, and the product was recovered by rotoevaporation of the chloroform.

The residues were then dissolved in isopropanol and the product was precipitated as a solid with the addition of an excess of ethyl ether. The solids were then collected by filtration and washed with ethyl ether. The solids were then slurried in acetone, and the acetone was removed by filtration. The acetone in the filtrate was then removed by rotoevaporation to yield an oil. Addition of 50 mL acetone and excess ether caused precipitation of solid product. The product was recrystallized by dissolving the solids in a minimum of hot methanol, adding some ethyl acetate, and rotoevaporating to remove a small amount of the methanol until a trace of solid was observed to precipitate. The recrystallization was then allowed to occur for two days at 0 C. NMR of the separate solid fractions indicated the solid which did not dissolve in the acetone is about 100% trans, and the remaining oily product recovered from the acetone is about 80/20 cis/trans. This indicates the cis isomer easily dissolves in acetone, while the trans isomer possesses limited solubility in acetone.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, and also to enable any person skilled in the art to make and use the inventions Many modifications of the exemplary embodiments of the invention disclosed above will readily occur to those skilled in the art. Accordingly, the invention is to be construed as including all structure and methods that fall within the scope of the appended claims.

1. A process for synthesizing trans-N,N-diethyldecahydroquinolinium cation or a mixture of cis-N)N-diethyldecahydroquinolinium cation and trans-N,N-diethyldecahydroquinolinium cation comprising:
a. hydrogenating quinoline in the presence of acetic acid, sulfuric acid and a platinum oxide catalyst to form a mixture of cis-decahydroquinoline and trans-decahydroquinoline;
b. recovering the mixture of cis-decahydroquinoline and trans-decahydroquinoline and freezing it;
c. thawing the frozen mixture of cis-decahydroquinoline and trans-decahydroquinoline until it separates into a liquid fraction and a solid fraction;
d. recovering the liquid fraction from step (c);
e. reacting the liquid fraction with ethyl halide to form a solid product which is a mixture of cis-N,N-diethyldecahydroquinolinium cation and trans-N,N-diethyldecahydroquinolinium cation;
f. recovering the solid product of step (e);
g. slurrying the solid product in acetone to selectively recover a precipitate with an increased amount of trans-N,N,-diethyldecahydroquinolinium cation and a remaining oily product recovered from the acetone having an increased amount of cis-N,N-diethyldecahydroquinolinium cation.

2. The process of claim 1, additionally including removing the acetone from the slurry by filtration.

3. The process of claim 1, additionally including the steps of,
a. removing the acetone from the slurry by filtration;
b. removing the acetone from the filtrate to yield an oil product;
c. adding acetone and ethyl ether to the oil to cause a solid to precipitate; and
d. recrystallizing and recovering the precipitate and a remaining oily product recovered from the acetone.

4. The process of claim 1, wherein the precipitate is at least 80% trans-N,N-diethyldecahydroquinolinium cation.

5. The process of claim 1, wherein the remaining oily product recovered from the acetone has a trans/cis ratio of between about 10/90 to about 40/60.

6. The process of claim 5, wherein the remaining oily product recovered from the acetone has a trans/cis ratio of between about 10/90 to about 30/70.

7. The process of claim 3, wherein the recrystallizing includes dissolving the solid from step (j) in a minimum of hot alcohol, adding some acetate, and removing some of the alcohol until a solid is observed to precipitate.

8. The process of claim 1, wherein the mixture of step (a) has less than 50% cis isomers.

9. The process of claim 8, wherein the mixture of step (a) has at least 25% cis isomers.

10. A method for synthesizing preferred isomer fractions of NN-diethyldecahydroquinolinium cation, comprising.
a. forming a mixture of cis-decahydroquinoline and trans-decahydroquinoline, wherein the mixture has at least about 25% cis-decahydroquinoline;
b. freezing and thawing the mixture until it separates into a liquid fraction and a solid fraction, wherein the liquid fraction has greater than about 40% cis-decahydroquinoline;
c. reacting the liquid fraction with ethyl halide to form a solid product which is a mixture of cis-N,N-diethyldecahydroquinolinium cation and trans-N,N-diethyldecahydroquinolinium cation;
d. recovering the solid product of step (c);
e. slurrying the solid product in acetone to selectively recover a precipitate with an increased amount of trans-N,N,-diethyldecahydroquinolinium cation and a remaining oily product recovered from the acetone having at least about 60% cis-N,N-diethyldecahydroquinolinium cation.

11. The method of claim 10, wherein the remaining oily product recovered from the acetone has at least about 70% cis-N,N-diethyldecahydroquinolinium cation.

12. The method of claim 10, wherein the remaining oily product recovered from the acetone has at least about 60% to about 90% cis-N,N-diethyldecahydroquinolinium cation.

13. The method of claim 12, wherein the remaining oily product recovered from the acetone has at least about 70% to about 90% cis-N,N-diethyldecahydroquinolinium cation.

14. The method of claim 10, wherein the recovering step (d) includes extracting with chloroform.

15. The method of claim 10, wherein the liquid fraction has greater than about 60% cis-decahydroquinoline.

16. A procedure for synthesizing preferred isomer fractions of N,N-diethyldecahydroquinolinium cation, comprising:
a. freezing and thawing a mixture of cis-decahydroquinoline and trans-decahydroquinoline, wherein the mixture has at least about 25% cis-decahydroquinoline, until the mixture separates into a liquid fraction having greater than about 40% cis-decahydroquinoline and a solid fraction;
b. reacting the liquid fraction with ethyl halide to form a solid product which is a mixture of cis-N,N-diethyldecahydroquinolinium cation and trans-N,N-diethyldecahydroquinolinium cation,
c. recovering the solid product of step (b) by filtering and extracting the filtrate with a halogenated hydrocarbon solvent;
d. slurrying the solid product in acetone to selectively recover a precipitate with an increased amount of trans-N,N,-diethyldecahydroquinolinium cation and a remaining oily product recovered from the acetone having at least about 60% cis-N,N-diethyldecahydroquinolinium cation.

17. The procedure of claim 16 wherein the remaining oily product recovered from the acetone has at least about 70% cis-N,N-diethyldecahydroquinolinium cation.

18. The procedure of claim 16, wherein the remaining oily product recovered from the acetone has at least about 60% to about 90% cis-N N-diethyldecahydroquinolinium cation.

19. The procedure of claim 16, wherein the remaining oily product recovered from the acetone has at least about 70% to about 90% cis-N,N-diethyldecahydroquinolinium cation.

20. The procedure of claim 16, wherein the halogenated hydrocarbon solvent is chloroform.
